SEQUENCE LISTING (15 sequences)

SEQ ID NO: 1 - protein, 4 aa, artificial sequence
  biodegradable spacer
GFLG

SEQ ID NO: 2 - protein, 3 aa, artificial sequence
  biodegradable spacer
GLG

SEQ ID NO: 3 - protein, 3 aa, artificial sequence
  biodegradable spacer
GVG

SEQ ID NO: 4 - protein, 3 aa, artificial sequence
  biodegradable spacer
GFA

SEQ ID NO: 5 - protein, 3 aa, artificial sequence
  biodegradable spacer
GLF

SEQ ID NO: 6 - protein, 3 aa, artificial sequence
  biodegradable spacer
GLA

SEQ ID NO: 7 - protein, 3 aa, artificial sequence
  biodegradable spacer
AVA

SEQ ID NO: 8 - protein, 4 aa, artificial sequence
  biodegradable spacer
GFFL

SEQ ID NO: 9 - protein, 4 aa, artificial sequence
  biodegradable spacer
GLLG

SEQ ID NO: 10 - protein, 5 aa, artificial sequence
  biodegradable spacer
GFAYA

SEQ ID NO: 11 - protein, 4 aa, artificial sequence
  biodegradable spacer
GFGF

SEQ ID NO: 12 - protein, 4 aa, artificial sequence
  biodegradable spacer
AGVF

SEQ ID NO: 13 - protein, 4 aa, artificial sequence
  biodegradable spacer
GFFG

SEQ ID NO: 14 - protein, 5 aa, artificial sequence
  biodegradable spacer
GFLGF

SEQ ID NO: 15 - protein, 6 aa, artificial sequence
  biodegradable spacer
GGFLGF

(19) United States
(12) Patent Application Publication
(10) Pub. No.: US 2007/0287680 A1
(43) Pub. Date: Dec. 13, 2007

(54) Combined Active and Passive Targeting of Biologically Active Agents

(75) Inventors: Vaikunth Cuchelkar, Salt Lake City, UT (US); Pavla Kopeckova, Salt Lake City, UT (US); C. Matthew Peterson, Bountiful, UT (US); Jindrich Kopecek, Salt Lake City, UT (US)

Correspondence Address: NEEDLE & ROSENBERG, P.C., SUITE 1000, 999 PEACHTREE STREET, ATLANTA, GA, 30309-3915, US

(73) Assignee: University of Utah Research Foundation, Salt Lake City, UT (US)

(21) Appl. No.: 11/547,872

(22) PCT Filed: May 5, 2005
(86) PCT No.: PCT/US05/15681

§ 371 Date: Jul. 11, 2007

Related U.S. Application Data
(60) Provisional application No. 60/569,770, filed on May 10, 2004.

(51) Int. Cl. C12N 15/01; A61K 31/7088; A61K 47/16; A61K 47/28; A61P 35/00

(52) U.S. Cl. 514/44; 435/455; 514/772; 514/773; 514/789

(57) ABSTRACT

Disclosed is a conjugate comprising a biologically active agent (drug) linked to a subcellular targeting moiety that targets a drug specifically to the nucleus. Targeting is achieved by attaching a steroid hormone (or an analog) to the drug. The steroid hormone attached to the drug binds its corresponding receptor, the formation of the receptor-ligand complex results in the internalization of the complex into the nucleus, thus resulting in nuclear translocation of the drug. Also disclosed is a conjugate (comprising the complex of the drug and the steroid hormone) bound to a polymer by spacers allowing for concurrent passive targeting to the tumor cell (afforded by attachment to the polymer by the EPR effect) and nuclear targeting of the conjugate (due to the presence of the steroid). Using a suitable degradable spacer allows for the release of free drug in the tumor and enhances nuclear targeting efficacy. The polymer can be further linked to a cellular targeting molecule, where the targeting molecule directs the polymer to specific cells. One may thus be able to effectively target drugs to the nucleus of tumor cells. With little or modifications, several therapeutic agents can be targeted using the invention.

### CROSS-REFERENCE TO RELATED APPLICATION

This application claims the benefit, under 35 U.S.C. §119(e) of U.S. Provisional Patent Application No. 60/569,770, filed on May 10, 2004, the contents of the entirety of which is incorporated by this reference.

### STATEMENT REGARDING FEDERALLY SPONSORED RESEARCH OR DEVELOPMENT

Work described herein was supported by National Institute of Health Grant #CA 51578. The United States government may have certain rights in the invention.

### TECHNICAL FIELD

This invention relates to biotechnology, more particularly to targeted delivery of biologically active agents such as drugs, prodrugs, proteinaceous molecules, genes, and/or nucleic acid sequences.

### BACKGROUND

Low molecular weight therapeutic agents diffuse throughout a cell and are not concentrated at a specific subcellular location. Targeting these agents to the subcellular site where they are most effective increases their efficacy. In addition, if such drugs are administered intravenously, they are systemically distributed to all tissues of the body. The action of these drugs at these unintended sites of distribution results in observable systemic side effects. It is thus preferred to localize the drug to the sites in the body where the action is desired.

Targeting of, for example, anticancer drugs in the case of tumors can be achieved by “passive targeting” and “active targeting”. Passive targeting is achieved by incorporating anticancer drugs into polymers. Active targeting is achieved by incorporating cellular targeting moieties that are specific to recognition molecules on the surface of the cells.

Polymers localize preferentially in solid tumors when compared to normal tissue. This occurs due to a phenomenon called the Enhanced Permeability and Retention (“EPR”) effect, which is attributed to morphological changes in tumor tissue, where the leaky vasculature produced due to neoangiogenesis results in the leakage of vascular contents into the extracellular tissue. In addition, the lymphatics may be blocked, which results in the accumulation of macromolecular agents in the extracellular tissue surrounding tumor cells (Matsumura Y, Maeda H, Cancer Res 4(12 Pt 1) (1986) 6387-6392). This phenomenon can be used to target tumor cells by attaching drugs to the polymers. Since polymers localize around tumor cells, the drugs attached to the polymers are also available at higher concentrations around the tumor. Drugs attached to polymers are taken inside cells by endocytosis. However, since the drugs remain attached to the polymer backbone, they may not be as effective as free drugs. This may be overcome by the use of biodegradable sequences to link the drug to the polymer backbone. The sequences that are chosen are such that they can be degraded inside the cell under specific conditions.

Polymer-based therapeutics have greater hydrodynamic volume in comparison to the free drug, which translates into a longer intravascular half-life. Polymer-based therapeutics also enhance the solubility and the bioavailability of insoluble drugs. Macromolecular therapeutics for anticancer drugs also can overcome certain cases of drug resistance. Other advantages afforded by polymer-based therapeutics include increased maximum tolerated dose, decreased non-specific toxicity, enhanced induction of apoptosis, and activation of alternate signaling pathways (Kopecek et al., Advances in Polymer Science 122 (Biopolymers II) (1995) 55-123).

In addition, cancer cells often have surface molecules that are either absent in normal tissue or over-expressed in comparison to the normal tissue. These may include growth factor receptors and/or certain antigens. Attaching recognition molecules to polymers that bind these molecules results in a high concentration of the polymers in the local environment of the tumor. Such targeting moieties include antibodies and ligands for cell surface receptors. Receptor mediated endocytosis initiated by the binding of some of these recognition molecules to their receptors can result in an increased intracellular concentration and correspondingly an enhanced effect.

PCT International Publication WO 00/11018A1 “Conjugates Of DNA Interacting Groups With Steroid Hormones For Use As Nucleic Acid Transfection Agents”, published Mar. 2, 2000, (the contents of which are incorporated by this reference) discloses compounds comprising a steroid hormone (e.g., an androgen, estrogen, or glucocorticoid) linked to a DNA-interacting molecule that target nucleic acids to the cell nucleus (e.g., an intercalating agent, cross-linking agent, or psoralen) and a method of introducing nucleic acids into the nucleus of cells with the help of such compounds. Similar work is disclosed in Rebuffat et al. “Selective enhancement of gene transfer by steroid-mediated gene delivery” Nature Biotechnology, 19:1155-1161 (2001), the contents of which is also incorporated by this reference.

### SUMMARY OF THE INVENTION

Disclosed herein is a method of targeting drugs specifically to the nucleus of cells. This targeting is achieved by attaching a steroid hormone or an analog to the biologically active agent. The steroid hormone attached to the biologically active agent binds its corresponding receptor; the formation of the receptor-ligand complex results in the internalization of the complex into the nucleus, thus resulting in nuclear localization of the biologically active agent. Biologically active agents may thus be targeted to the nucleus of the cell. With or without minor modifications, several therapeutic agents can be targeted using the invention in consideration.

The invention includes a polymeric delivery system for biologically active agents with concurrent nuclear targeting. Biologically active agents are modified by attaching a steroid hormone thereto.

Such biologically active agent-steroid derivatives are further targeted to the tumor tissue by attaching them to a polymer (for the EPR effect) with a biodegradable sequence. The biodegradable sequences selected are ones that can be degraded by enzymes present inside the cell (especially the lysosomes) to link the drugs to the polymer (Duncan et al., Makromolecular Chemie 184 1997-2008 (1983)). An example of such a biodegradable sequence is Gly-Phe-Leu-Gly (SEQ ID NO:1) that is degraded by Cathepsin B in lysosomes. When such macromolecular agents are taken inside the cell by endocytosis they localize within the lysosomes. The biodegradable sequences can then be degraded by the specific enzyme inside the lysosomes resulting in the release of the free biologically active agent. The therapeutic effect afforded by using this approach is better than that in the case of biologically active agents attached to the polymer by non-degradable sequences. By using this system, we will achieve targeting to the cancer and then in addition be able to localize the free drug to the nucleus of the cells. It is expected that this approach will greatly enhance the therapeutic efficacy of the biologically active agent. This will translate into lower doses being administered.

The invention also includes a “double-targeted polymeric delivery system”. In such an instance, the biologically active agent will be modified by attaching a steroid hormone as the nuclear targeting signal. The biologically active agent-steroid derivative will be attached to the polymer by a biodegradable sequence. The double-targeted system also includes attaching a cellular targeting moiety such as an antibody (e.g., polyclonal antibody, monoclonal antibody, phage display antibody, ribosome display, or antibody fragment) to the polymer. The attachment of the cellular targeting moiety like the antibody is expected to result in enhanced uptake by tumor cells. This effect, when combined with the potential to deliver the therapeutic agent in high concentrations to the nucleus due to nuclear targeting using the steroid hormone, will result in an unexpectedly high biological activity using this approach.

The use of the double targeting system will ensure that only the cells that express the surface recognition moiety will be targeted. Use of subcellular targeting moieties will further enable a reduction of the dose of the drug that will need to be administered. This will ensure that only the right cells will be killed with a very small amount of drug. This delivery system has great potential in the delivery of therapeutic agents for the treatment of cancer. The use of cellular signaling pathways ensures that this strategy will work much more effectively in cells, which express the particular steroid hormone receptor—this affords another element of specificity to the whole approach.

Potential applications of the nuclear-targeted polymeric conjugates are numerous. Certain anticancer drugs are most effective in the nucleus. Anticancer drugs that act on the DNA to elicit their cytotoxic effect would be greatly benefited by using this approach. Agents used in the photodynamic therapy of cancer would also see an increase in the therapeutic effect following targeting to the nucleus since the nucleus is hypersensitive to photodynamic damage. Another field in which this invention would prove useful would be in gene therapy. Gene therapy requires that genes be delivered to the nucleus effectively; this invention fulfils that need. Other agents that can be targeted similarly are nucleic acid sequences (e.g., DNA or RNA) of epitopes for vaccine production.

It is to be understood that the drugs or genes (as therapeutic agents) or antibodies (as targeting moieties) or Gly-Phe-Leu-Gly (SEQ ID NO:1) (as biodegradable linker sequences) as mentioned below are merely illustrative of the numerous agents that could be used.

### BRIEF DESCRIPTION OF THE DRAWINGS

FIG. 1 depicts a general example of this system; wherein “P” is an inert polymeric backbone of the delivery system and connector “C” is linked to the polymer backbone P via a spacer (“S1”). “D” is a therapeutic moiety linked to one arm of the connector C. “N” is a nuclear targeting moiety linked to the other arm of the connector C. “T” is a tissue-targeting moiety linked to the polymer backbone P via a spacer “S3”. “L” is an optional bioassay label linked to the polymer backbone via a spacer “S2”. “X” is an optional biodegradable cross linkage in the delivery system.

FIG. 2 depicts some examples of hormone-drug conjugates and their polymeric delivery systems. In A, an example is given demonstrating a norethisterone (NET)-targeted system for the delivery of doxorubicin (Dox). In this case, the connector used is Tyrosine (Tyr). The connector is linked to the poly-L-lysine (PLL) polymeric backbone via a degradable glycylleucylglycyl (GLG) spacer. In addition, the polymer also bears a LHRH moiety to target tissues overexpressing the LHRH receptor. In B, an example demonstrating a TTNPB (4-[(E)-2-(5,6,7,8-tetrahydro-5,5,8,8-tetramethyl-2-naphthalenyl)-1-propenyl]benzoic acid) targeted system for the delivery of 9-aminocamptothecin (9-AC). In this case, the connector used is glutamic acid (Glu). The connector is linked to the polymeric backbone (polyethylene glycol-co-aspartic acid) via a non-degradable glycylglycyl (GG) spacer. In addition, the polymer also bears an OV-TL 16 antibody to target ovarian cancer tissue specifically.

FIG. 3 graphically depicts a scheme for the synthesis of a double-targeted polymeric delivery system for mesochlorin.

FIG. 4 graphically illustrates the synthesis of Cort-ONp

FIG. 5 graphically illustrates the synthesis of Lys-Mce₆ intermediate FIG. 6 graphically illustrates the synthesis of Cort-Lys-Mce₆

FIG. 7 graphically illustrates the synthesis of P-GFLG-Cort-Lys-Mce₆

FIG. 8 demonstrates the biorecognition of Cort-Lys-Mce₆ derivative using the GFP-GR system. Cortisol and Cort-Lys-Mce₆ derivative is able to shuttle GFP tagged GR from the cytoplasm to the nucleus. Mce₆ by itself cannot cause redistribution of the GFP tagged GR.

FIG. 9 depicts the subcellular localization of free Mce₆ using confocal microscopy after a 24 hour incubation period at 37° C. with (A) Mce₆ (red) and (B) DAPI (nuclear marker). DIC image is window (C). Combined image (D) shows a faint Mce₆ fluorescence in the nucleus.

FIG. 10 depicts the subcellular localization of Cort-Lys-Mce₆ derivative using confocal microscopy after 24 hours incubation at 37° C. with (A) Mce₆ (red) and (B) DAPI (nuclear marker). DIC image is window (C). Combined image (D) shows colocalization of Mce₆ and DAPI indicating presence of Colt-Lys-Mce₆ in the nucleus

### DETAILED DESCRIPTION OF THE INVENTION

When low molecular weight compounds are administered to a subject (e.g., a mammal such as a human patient), they are distributed all over the body and enter cells by the process of diffusion. This process results in the compounds' presence in virtually every cell of the body. Action of these drugs at the unintended sites in the body results in side effects. Side effects are especially important in the case of anti-cancer drugs. Anti-cancer drugs are generally low molecular weight cytotoxic/cytostatic drugs that are administered intravenously. This cytotoxic/cytostatic action of the drug in normal tissues results in the side effects often seen with anticancer drugs. Side effects can be reduced by localizing the drugs effectively and specifically at the tumor site.

Attachment of low molecular weight therapeutic agents to polymeric carriers has been extensively studied as a tool to improve their specificity for tumor tissue. Polymer-drug conjugates accumulate preferentially in the tumor tissue passively due to the aforementioned EPR effect. Macromolecular therapeutics for anticancer drugs also can overcome certain cases of drug resistance. Macromolecular therapeutic agents also afford other advantages in contrast to low molecular weight drugs: increased half life, increased maximum tolerated dose, decreased non-specific toxicity, targetability (by the attachment of ligands to the polymer that are specific to receptors on the cell surface), increased solubility and bioavailability, enhanced induction of apoptosis, and activation of alternate signaling pathways. To target the tumor cells specifically, antibodies and ligands for cell surface receptors (growth factor molecules and hormones for example) can be attached to the polymer backbone. Attaching such targeting moieties can also lead to internalization of the polymer conjugate by receptor-mediated endocytosis and an enhanced intracellular concentration.

Since free drug is more effective in comparison to polymer-bound therapeutics, the polymer-drug conjugates are designed such that they are stable in the blood stream, but release the drug in tumor tissue. This can be done by incorporating degradable spacers to link the drug to the polymer backbone. Biodegradable spacers like glycylphenylalanylleucylglycine (GFLG) (SEQ ID NO:1) are stable in the blood stream, but are broken down by proteases like cathepsin B in the lysosomes. The strategy of using the GFLG spacer to link the drug to the polymer enables us to exploit the advantages afforded by polymers as well as releasing the active drug inside the cell to obtain maximum therapeutic effect. Other biodegradable spacers can be used depending on specific requirements. Such a polymeric delivery system can be used to efficiently and specifically deliver therapeutic agents to the tumor tissue.

The distribution of low molecular weight therapeutic agents inside the cell depends on the mode of entry into the cell, its physico-chemical properties and its subsequent redistribution. Since the mode of entry is mainly via diffusion, free drug is not concentrated at a specific subcellular location. Targeting the free drug to the subcellular site where is it most effective increases its therapeutic efficacy. For example, photodynamic therapy is a therapeutic modality for cancer, which involves the administration of photosensitizers to the patient followed by their activation by illumination with light of a specific wavelength. On activation, the photosensitizers are excited to their singlet state—however, this state is unstable and the excited photosensitizer decays rapidly and gives off excess energy. This energy is transferred to molecular oxygen in the environment, forming reactive oxygen species, the most important of which is singlet oxygen (¹O₂). Singlet oxygen is highly reactive and nonspecifically reacts with biomolecules present in the surrounding leading to cellular damage. However, singlet oxygen has a short half-life (about 4 μs) in the aqueous conditions and very limited diffusion capability. This results in cellular damage induced by singlet oxygen, localized to about 100 nm around the site of its generation. The nucleus is known to be hypersensitive to photodynamic therapy induced damage (Peng et al., Ultrastructural Pathology 20(2) (1996) 109-129). However, most photosensitizers do not target this subcellular compartment. Singlet oxygen produces single strand breaks and base modifications in the DNA. Inactivation of enzymes in DNA repair produced by singlet oxygen further hampers the ability of the cell to repair the damage produced. Accumulation of cellular damage induced by the singlet oxygen eventually leads to cell death. Studies targeting the photosensitizer chlorine e6 to the nucleus using various strategies have been shown to increase cytotoxicity (Akhlynina et al., Journal of Biological Chemistry, 272(33):20328-20331 (1997) and Bisland et al., Bioconjugate Chemistry, 10(6) 982-992 (1999)). Thus, targeting the nucleus is an attractive method to increase the cytotoxicity of existing photosensitizers. This targeting can be achieved by the use of steroid hormones (or their analogs) as nuclear targeting moieties.

Steroid hormones are known to exert their actions at the level of the cell by two mechanisms. The “rapid action” is facilitated by membrane surface receptors, while the classical mechanism of action is much slower and is exerted at the DNA level by means of cytosolic steroid hormone receptors (SHR). This genomic action is mediated by the formation of the steroid hormone-receptor complex in the cytoplasm followed by the shuttling of this complex from the cytoplasm to the nucleus through the nuclear pore complex (NPC) (Mangelsdorf et al., Cell, 83(6):835-839 (1995)). This movement of the steroid hormone-receptor complex across the nuclear membrane occurs via nuclear localization signals (NLS) present on the receptor, which are exposed following binding of the steroid hormone. AR (androgen receptor) contains a bipartite NLS while PR (progesterone receptor) and GR (glucocorticoid receptor) contain tripartite NLSs in their DNA binding domains. These classical NLSs present on the SHRs are imported into the nucleus via the importin alpha mediated import pathway. GR and glucocorticoid analogs have been used previously as a transport mechanism from the cytoplasm to the nucleus.

Disclosed herein is a strategy to target a cell nucleus with therapeutic agents thus increasing their cytotoxic effect. Specifically, presented, among other things, is an example for the nuclear targeting of photosensitizers for photodynamic therapy of cancer. Nuclear targeting causes damage at the genomic level and induces apoptosis in the cancer cells leading to a relatively decreased inflammatory response. The decreased inflammatory response is one of the desirable clinical features to be achieved using this strategy. It is expected that nuclear targeting will cause a similar increase in the efficacy of other therapeutic agents that are known to act at the level of the nucleus.

As an example, the synthesis of a double-targeted polymeric delivery system for Mesochlorin e₆ monoethylene diamine disodium salt (20-[(2-Aminoethylcarbamoyl)-methyl]-18-(2-carboxy-ethyl)-7,12-diethyl-3,13,17-trimethyl-17,18,21,23-tetrahydro-porphine-2-carboxylic acid disodium salt) (Mce₆—an agent used in the photodynamic therapy of cancer) is disclosed that targets the nucleus. Cortisol (a glucocorticoid analog) was used as the nuclear targeting moiety. The analysis of the structure of GR indicates that the structure of cortisol might be modified without impairment of binding to the receptor (Williams A P, Sigler P B, Nature, 393(6683):392-396 (1998)). Lysine was used to connect the cortisol and the Mce₆. Cortisol was bound to the alpha-NH₂ group, while Mce₆ was bound to the —COOH group of the lysine. This resulted in the formation of alpha-N-(Cortisol-C₁₇-carbamoyl)-Lysyl-Mesochlorin ethyl amide((20-{[2-(6-tert-Butoxycarbonylamino-2-(11,17-Dihydroxy-17-(2-hydroxy-acetyl)-10,13-dimethyl-1,2,6,7,8,9,10,11,12,13,14,15,16,17-tetradecahydrocyclopenta[a]phenanthren-3-one)-carbamoyl-hexanoylamino)-ethylcarbamoyl]-methyl}-18-(2-carboxy-ethyl)-7,12-diethyl-3,8,13,17-tetramethyl-17,18,21,23-tetrahydroporphine-2-carboxylic acid) that we designated as Cort-Lys-Mce₆ (Mce₆-cortisol derivative). We were able to demonstrate that Cort-Lys-Mce₆ was able to localize to the nucleus and also demonstrate an enhanced cytotoxicity.

The free epsilon-NH₂ group on the Cort-Lys-Mce₆ was then used to attach the Cort-Lys-Mce₆ to a PHPMA backbone bearing pendant ONp groups with biodegradable GFLG spacers. PGFLG-Cort-Lys-Mce₆ had an enhanced cytotoxicity as compared to PGFLG-Mce₆ due to the nuclear targeting. After the attachment of the Mce₆-cortisol derivative, the remaining ONp groups will be used to bind OV-TL16 antibody (which is specific to OA antigen on ovarian carcinoma cells). We are thus able to devise a polymeric delivery system for Mce₆ with OV-TL16 as the cellular targeting moiety and the steroid (cortisol) as the subcellular targeting moiety. This delivery system will be able to achieve enhanced uptake in the tumor cells and also target the mesochlorin to the nucleus (thereby increasing the efficacy). This is expected to greatly increase the therapeutic efficacy of this agent in ovarian cancer. Results of some of the experiments conducted with this system are detailed in the experimental section.

A similar strategy would also serve to enhance the efficacy of anticancer drugs that act at the level of the nucleus. Some such drugs include anticancer drugs like topoisomerase I inhibitors (for example, camptothecin, 9-aminocamptothecin, irinotecan, topotecan), anthracyclines (for example, doxorubicin, daunorubicin). Similarly, this strategy can also be used to deliver genes/antisense oligonucleotides (ODNs)/peptide nucleic acids (PNAs)/small interfering RNAs (siRNAs) to the nucleus—this would involve replacement of the drug with the desired gene/ODN/PNA/siRNA. Nucleic acid sequences of epitopes for vaccine production can also be targeted similarly and result in an increased efficacy and specificity.

This strategy is flexible in that it can be applied to a wide variety of cytosolic receptors. Some of the cytosolic receptors that can be used for the delivery of the molecules to the nucleus include the steroid hormone receptors (AR, PR, and ER), other nuclear receptors [Peroxisome Proliferation Activated Receptors (PPAR), Liver X receptors (LXR)] and various orphan receptors [Retinoid X receptors (RXR), Benzoate X receptor (BXR), Constitutive Androstane Receptor β (CARβ), Pregnane X receptor (PXR), Steroid and Xenobiotic receptor (SXR), Farnesoid X receptors (FXR)]. Ligands that are specific for these cytosolic receptors can be used in the place of cortisol as the subcellular targeting moiety. The cytosolic receptor can be so selected as to afford some selectivity in the type of cells that are targeted. Receptors that are specifically present in certain tissues can be used to provide nuclear targeting in such tissues only and thereby focus their effect. A list of some of the receptors that could be used and ligands that could be used for targeting such receptors in included in Appendix A.

Besides lysine, other molecules can serve to connect the three constituents of the system (drug, hormone, and polymer backbone). The connector preferably has three functional groups present that can react with the aforementioned constituents. Such can be fulfilled by several molecules besides lysine-like trifunctional amino acids (glutamic acid, aspartic acid, ornithine, cysteine, serine and tyrosine) and other multifunctional molecules.

Various polymeric carriers can serve as the backbone for the delivery of this system. Some of the polymeric carriers (other than HPMA copolymers) that are suitable include Poly(L-glutamic acid) (PGA), Poly(L-lysine) (PLL), PEG (Polyethylene glycol) and PEG-block copolymers (for example, polyethylene glycol-co-aspartic acid). Other polymeric carriers that could be used include polymers formed from monomeric units selected from the group including but not limited to N-(2-hydroxypropyl)methacrylamide, N-(2-hydroxyethyl)methacrylamide, N-isopropylacrylamide, acrylamide, N,N dimethylacrylamide, N-vinylpyrrolidone, vinyl alcohol, 2-methacryloxyethyl glucoside, acrylic acid, methacrylic acid, vinylphosphonic acid, styrenesulfonic acid, maleic acid, 2-methacryloxyethyltrimethylammonium chloride, methacrylamidopropyltrimethylammonium chloride, methacryloylcholine methyl sulfate, N-methylolacrylamide, 2-hydroxy-3-methacryloxypropyltrimethylammonium chloride, 2-methacryloxyethyltrimethylammonium bromide, 2-vinyl-1-methylpyridinium bromide, 4-vinyl-1-methylpyridinium bromide, ethyleneimine, (N-acetyl)ethyleneimine, (N-hydroxyethyl)ethyleneimine and allylamine. With suitable modifications, these polymer backbones can be used to attach the hormone-drug derivative via biodegradable spacers.

We can further enhance the efficacy and specificity of this system using cellular targeting moieties. Various moieties can be used to target certain tissues and cancers specifically. Antibodies that target specific antigens on the surface of cancer cells can be used to localize the polymeric conjugate to tumor tissue actively. Some such antigen-antibody combinations include OA3-OVTL16, Pgp-UIC2 and EGFR-anti-EGFR antibody. In addition to using antibodies to target specific tissues, various other ligands that are specific for receptors on the surface of the cells can be used. Some such ligand-receptor combinations include LHRH-LHRH receptor and insulin-insulin receptor. In addition, binding of the conjugates to the cells can result in an enhanced uptake due to receptor-mediated endocytosis. This results in an increase in the concentration of the drug inside the cell and further increases the effect. The examples of the cellular targeting moieties mentioned are examples of the general idea and do not limit the targeting molecules that can be used.

Various kinds of biodegradable spacers can be used in the polymeric delivery system. These may be peptide sequences made from L-amino acids (for example, Gly-Phe-Leu-Gly (SEQ ID NO:1), Gly-Leu-Gly (SEQ ID NO:2), Gly-Val-Gly (SEQ ID NO:3), Gly-Phe-Ala (SEQ ID NO:4), Gly-Leu-Phe (SEQ ID NO:5), Gly-Leu-Ala (SEQ ID NO:6), Ala-Val-Ala (SEQ ID NO:7), Gly-Phe-Phe-Leu (SEQ ID NO:8), Gly-Leu-Leu-Gly (SEQ ID NO:9), Gly-Phe-Tyr-Ala (SEQ ID NO:10), Gly-Phe-Gly-Phe (SEQ ID NO:11), Ala-Gly-Val-Phe (SEQ ID NO:12), Gly-Phe-Phe-Gly (SEQ ID NO:13), Gly-Phe-Leu-Gly-Phe (SEQ ID NO:14), and Gly-Gly-Phe-Leu-Gly-Phe (SEQ ID NO:15)), spacers that undergo 1,6 elimination, pH sensitive bonds or disulfide bonds. The type of spacer that will be used will depend on the individual circumstance and the expected mechanism of action. For example, protein transduction domains can be used to achieve increased concentration in the cells is another method to increase intracellular concentrations—however, it is to be realized that if this approach is followed, the conjugate will bypass the traditional endocytic pathway and hence the use of lysosomally degradable sequences may not be the most optimal approach. In this case, we can use other sequences like disulfide (S—S) bonds that can be reduced in the cytoplasm to link the hormone-drug conjugate to the polymer backbone.

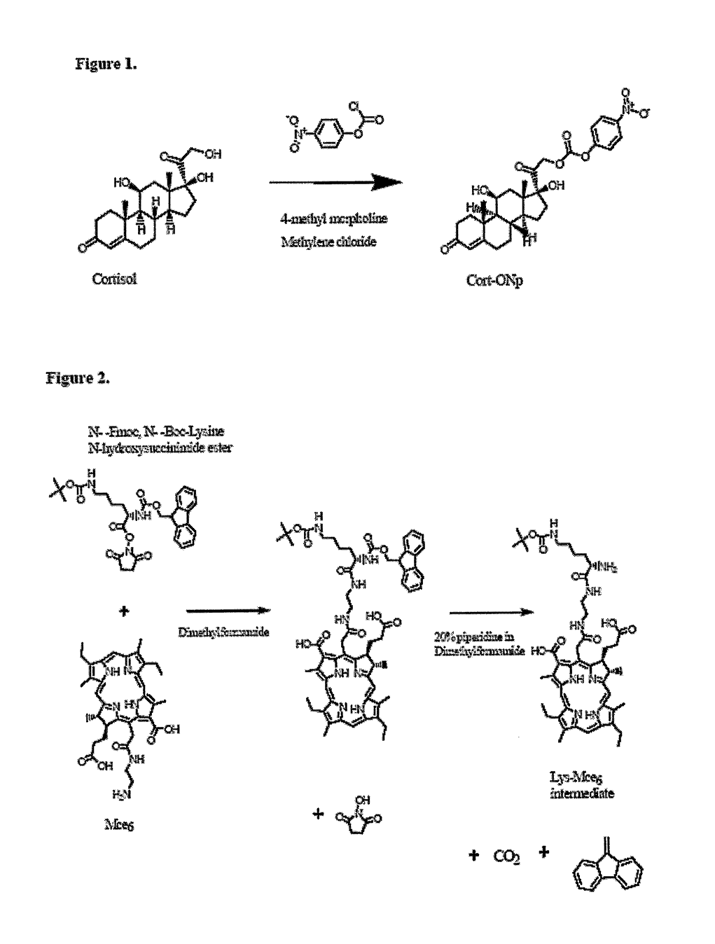

For the synthesis of these variations of the system, it is not necessary to perform the synthesis in the same order as described for P-GFLG-Cort-Lys-Mce6. The sequence that will be followed will typically depend on the individual circumstances. Some steps may be carried out earlier to ensure the stability of the various constituents of the system. In general, our strategy is summarized as shown in FIG. 1. In FIG. 1, C is the connector that is linked to the water-soluble inert polymer backbone (P) via a spacer (S1), which may be biodegradable or non-biodegradable. D is the therapeutic agent (anticancer drug/genes/ODN/PNA/siRNA) bonded to one arm of the connector C. N is the nuclear targeting moiety (ligand specific for nuclear receptor) bonded to the other arm of the connector C. T is the optional tissue targeting moiety covalently bound to the polymer backbone (P) via biodegradable or non-degradable spacer (S3); L is an optional bio-assay label covalently bonded to the polymer backbone (P) via a non-degradable spacer (S2) which can be the same or different than S1 or S2 when they are non-degradable; and X is an optional biodegradable cross-linkage between two polymer chains (P).

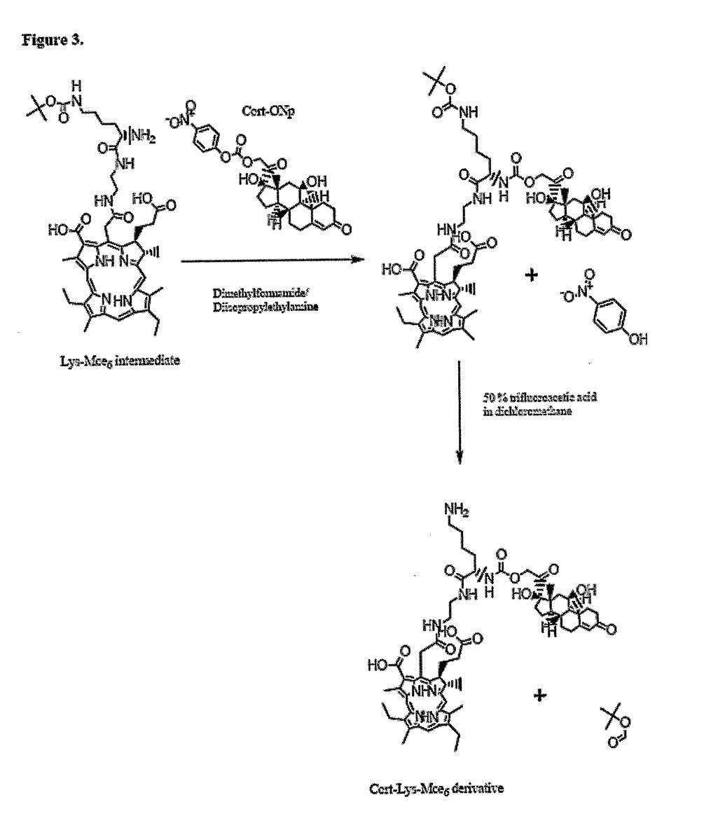

Some of the possible hormone-drug conjugates and the polymeric delivery systems for their delivery are shown in FIG. 2. It is to be understood that these are simply illustrations of the general principle, and alterations and modifications of the invention are within the scope of the invention.

The first example considered is that of a norethisterone (NET) targeted conjugate of the anthracycline anti-cancer drug, doxorubicin (Dox) and the polymeric delivery system for the delivery of the NET-Dox conjugate. The connector used in this case is tyrosine (Tyr). NET is a progesterone analog that will interact with the progesterone receptor (PR) and facilitate transport of the NET-Tyr-Dox into the nucleus. The connector Tyr is linked to the polymer backbone via a biodegradable glycylleucylglycyl (GLG) spacer.

The system in consideration also actively targets tissues overexpressing the LHRH receptor by using an LHRH moiety. The LHRH is connected to the poly-L-lysine (PLL) polymer backbone via a non-degradable glycylglycyl (GG) spacer. The monomeric structure with the NET-Tyr-Dox attached by the GLG spacer can range from 0.01-80 mole % of the polymer backbone. The content of the LHRH connected to the PLL backbone by a GG spacer can range from 0.01 to 20 mole % of the polymer backbone.

The second example uses glutamic acid (Glu) as a connector. In this case, TTNPB (4-[(E)-2-(5,6,7,8-tetrahydro-5,5,8,8-tetramethyl-2-naphthalenyl)-1-propenyl]benzoic acid) has been used to target the nucleus via the Farnesoid X receptor (FXR). The drug used in this case is a Topoisomerase I inhibitor, 9-aminocamptothecin (9-AC). The TTNPB-Glu-9-AC conjugate is linked covalently to the polymer backbone via a nondegradable GG spacer. In addition, the polymer also bears an OV-TL 16 antibody to target ovarian cancer tissue specifically (which overexpresses the OA3 antigen that is recognized by OV-TL16). The OV-TL16 antibody is linked to the polymer backbone (which is a copolymer of polyethylene glycol and aspartic acid) (PEG-co-Asp) via a nondegradable GG spacer. The monomeric structure with the TTNPB-Glu-9-AC conjugate attached by the GG spacer can range from 0.01-90 mole % of the polymer backbone. A higher drug loading content can be achieved in this case because the polymer backbone is extremely hydrophilic. The content of the OV-TL16 antibody connected to the PEG-co-Asp backbone can range from 0.01 to 50 mole % of the polymer backbone.

The invention is further explained by the use of the following illustrative examples. These examples will enable those skilled in the art to more clearly understand how to practice the present invention. It has to be understood that, while the invention has been described in conjunction with the preferred specific embodiments thereof, that which follows is intended to illustrate and not limit the scope of the invention. Other aspects of the invention will be apparent to those skilled in the art to which the invention pertains.

### EXAMPLES

### Example I

### Synthesis of Cort-Lys-Mce₆

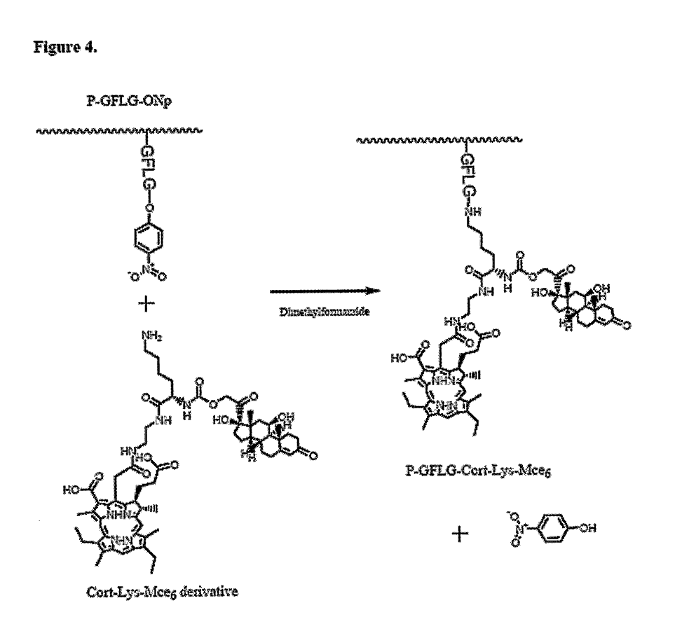

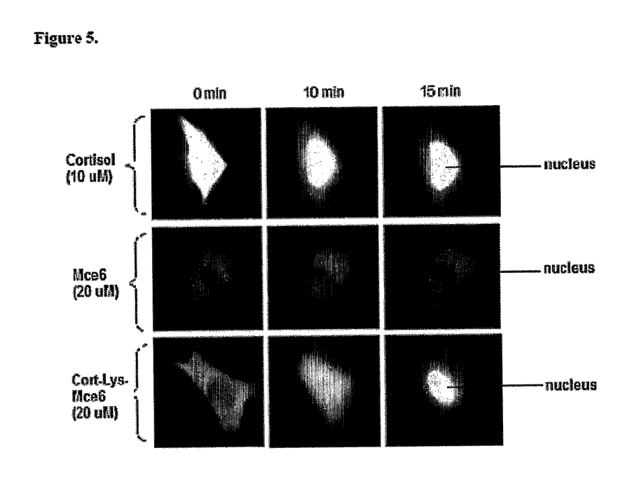

The example involves the synthesis of a double-targeted system for the nuclear targeting of Mce₆ (FIG. 3) and illustrates the synthetic process involved in the synthesis of Cortisol-Lys-Mce₆. Cortisol was acylated with twice molar excess of 4-nitrophenyl chloroformate in methylene chloride and thrice molar excess of 4-methyl morpholine to form Cort-ONp (Cortisol-C17-4-nitrophenyl ester) (carbonic acid 2-(11,17-dihydroxy-10,13-dimethyl-3-oxo-2,3,6,7,8,9,10,11,12,13,14,15,16,17-tetradecahydro-1H-cyclopenta[a]phenanthren-17-yl)-2-oxo-ethyl ester 4-nitro-phenyl ester) (FIG. 4). The reaction mixture was washed sequentially with 1 N hydrochloric acid, concentrated solution of sodium bicarbonate and concentrated solution of sodium chloride. The solution was then dried over sodium sulfate and crystallized to obtain the product (Cort-ONp).

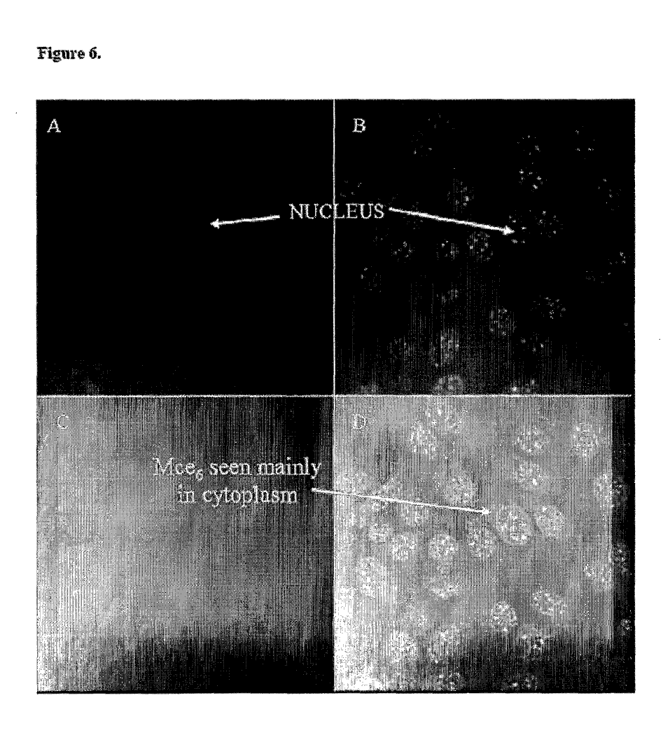

Mce₆ was reacted with N-alpha-Fmoc, N-ε-Boc-Lysine N-hydroxysuccinimide ester(6-tert-Butoxycarbonylamino-2-(9H-fluoren-9-ylmethoxy-carbonylamino)-hexanoic acid 2,5-dioxo-pyrrolidin-1-yl ester) in dimethylfommamide (DMF) followed by the deprotection of the Fmoc group using 20% piperidine in DMF for 30 minutes (FIG. 5) to produce the Lys-Mce₆ intermediate (alpha-NH₂,N-ε-Boc-Lysyl-Mesochlorin ethyl amide or 20-{[2-(2-Amino-6-tert-butoxycarbonylamino-hexanoylamino)-ethylcarbamoyl]-methyl}-18-(2-carboxy-ethyl)-7,12-diethyl-3,8,13,17-tetramethyl-17,18,21,23-tetrahydro-porphine-2-carboxylic acid) (FIG. 5). Next, the reactive ONp group on Cort-ONp was aminolysed with the alpha-NH₂ of the lysine of the Lys-Mce₆ intermediate in DMF with DIPEA (Diisopropylethylamine). The reaction mixture was purified by column chromatography using Sephadex LH-20 beads equilibrated in methanol. The major fraction was collected and verified to be the product by mass spectrometry. The N-ε-Boc group of lysine of the product was deprotected with 50% trifluoroacetic acid (TFA) in methylene chloride to obtain the Cort-Lys-Mce₆ derivative (FIG. 6). The product was purified using preparative HPLC/RPC in acetonitrile/H₂O. The product was characterized by TLC, UV spectroscopy, elemental analysis and mass spectrometry.

### Example II

### Synthesis of P-GFLG-Cort-Lys-Mce₆

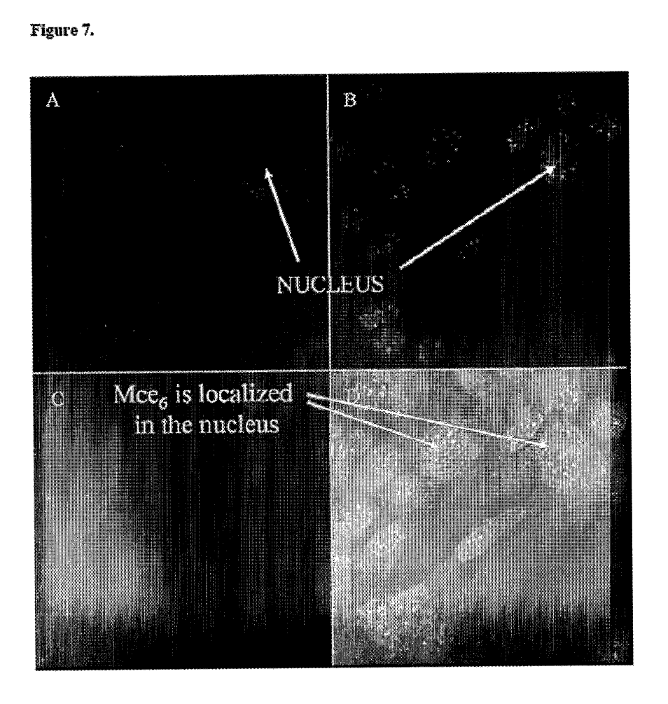

The polymer precursor P-GFLG-ONp (P=PHPMA backbone), was prepared by radical precipitation copolymerization of HPMA (2-hydroxypropylmethacrylamide) and N-methacryloylglycylphenylalanylleucylglycyl p-nitrophenyl ester in acetone in the presence of 2,2′-azobisisobutyronitrile (AIBN). Next, the binding of Cort-Lys-Mce₆ to P-GFLG-ONp was performed in DMF (FIG. 7). Unreacted 4-nitrophenoxy group was eliminated by the addition of 1-amino-2-propanol. The product was isolated by precipitation into acetone and purified by chromatography in methanol. The polymer was then dialyzed against deionized water and isolated by freeze-drying.

### Example III

### Synthesis of Control Polymers

The control polymer P-GFLG-Mce₆ was synthesized by the aminolysis of P-GFLG-ONp with free Mce₆ in DMF followed by the elimination of unreacted p-nitrophenoxy group with 1-amino-2-propanol. The polymer was then precipitated in acetone and purified by column chromatography in methanol. Dialysis was then performed in deionized water followed by lyophilization.

### Example IV

### Characterization of Polymer Conjugates

The molecular weight of the conjugates was determined by size exclusion chromatography using the AKTA FPLC system (Pharmacia) on a high resolution Superose 6 (HR 10/30) column calibrated with PHPMA samples in buffer 30% acetonitrile in PBS. The absolute molecular weight was determined using laser light scattering detector in combination with refractive index detection. The content of Mce₆ in the polymer was determined by UV spectroscopy in methanol (ε=158000 M⁻¹ cm⁻¹ in methanol at 395 nm).

Cort-Lys-Mce₆ was covalently bound to an N-(2-hydroxypropyl)-methacrylamide (“HPMA”) copolymer backbone via a biodegradable glycylphenylalanylleucylglycine (“GFLG”) spacer. Characterization of the HPMA copolymer-bound Cort-Lys-Mce₆ (P-GFLG-Cort-Lys-Mce₆) revealed 1.3 mol % drug-terminated side chains as determined by UV spectrometry. The weight average molecular weight, M_{w}, of the conjugate was determined to be approximately 22 kDa by size-exclusion chromatography. The polydispersity (M_{w}/Mₙ) was approximately 1.55. For the P-GFLG-Mce₆, UV spectrophotometry revealed 3.5 mol % drug-terminated side chains. The weight average molecular weight, M_{w}, of the conjugate was determined to be approximately 27 kDa. The polydispersity (M_{w}/Mₙ) was approximately 1.35.

### Example V

### Biorecognition of Cort-Lys-Mce₆

To demonstrate the ability of Cort-Lys-Mce₆ to bind the glucocorticoid receptor (GR), the green fluorescent protein tagged-GR (GFP-GR) system was utilized. This system involves the generation of GFP labeled GR in the cell. The cells are then treated with the drug and the change in the localization of the fluorescently tagged GR is followed. Cells were transfected with GFP-GR plasmids as previously. Briefly, 5×10⁶ cells were transfected with 2 μg of the GFP-GR plasmid plus carrier DNA using an Electrosquare porator ECM 830 system (BTX, San Diego, Calif.). The electroporation was performed using 3 pulses each of 135 V for 10 milliseconds, and 3 pulses. Cells were allowed to recover on ice for 5 minutes and were then diluted with phenol red-free DMEM with 10% charcoal stripped FBS and plated on a clear cover glass (Corning no. 1, 22 mm²) into live cell chambers (Lab-Tek II chambered cover glass with cover). These cells were incubated in cell culture conditions for 24 hrs. Prior to microscopy, the media was replaced and Cortisol (positive control), Mce₆ (negative control) and Cort-Lys-Mce₆ (test substance) solutions in DMEM with 10% charcoal stripped FBS were then added to cell chambers at appropriate doses. The import of GFP-receptor-conjugate complexes was monitored using an Olympus IX701F inverted fluorescence microscope and an EGFP filter set (Chroma Technology Corp.) (exciter 480 nm, emitter 510 nm, beam splitter 495 nm). Cells were maintained at 37° C. using a Nevtek ASI 400 Air Stream Incubator (variable temperature control) attached to a temperature probe as previously. Cells were photographed using an F-View Monochrome CCD camera.

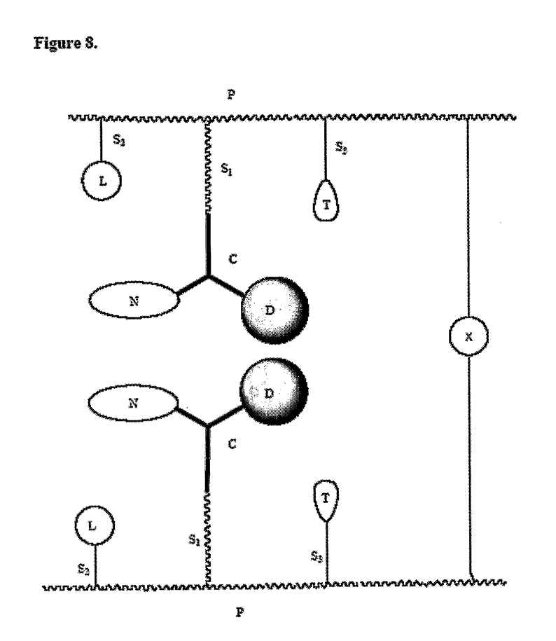

Cells treated with cortisol and Cort-Lys-Mce₆ demonstrated an increase in the fluorescence level in the nucleus and accompanied by a decrease in the cytoplasmic fluorescence (FIG. 8). This redistribution was both time- and concentration-dependent.

In contrast, Mce₆ used at higher concentrations (20 μM and 30 μM) did not induce any change in the localization of the receptors following treatment (FIG. 8). Images taken up to 30 minutes after treatment did not any change in the original cytoplasmic localization of GFP-GR. These experiments demonstrate the ability of the cortisol moiety on the Cort-Lys-Mce₆ derivative to recognize and bind the GR.

The ability of the Cort-Lys-Mce₆ to bind the GR and induce nuclear translocation was independently verified using a MMTV luciferase assay (data not shown).

### Example VI

### Confocal Fluorescence Microscopy to Demonstrate Subcellular Localization of Cort-Lys-Mce₆

Subcellular localization was determined using confocal fluorescence microscopy. 150000 cells were seeded on previously sterilized glass coverslips in 35 mm dishes 24 hours before incubation with the drug. The cells were incubated with the drug (free Mce₆, Cort-Lys-Mce₆ or HPMA copolymer-bound Mce₆ conjugates) for 24 hours and the cells were washed twice with DPBS (Dulbeco's Phosphate Buffered Saline) after incubation. The cells were then fixed with 3% paraformaldehyde for 20 minutes at room temperature. DAPI (4′,6-diamidino-2-phenylindole) (600 nM) was incubated with the cells and used as a nuclear marker. The cells were mounted with SlowFade Light Antifade medium (Molecular Probes, Eugene, Oreg.) and sealed as described previously. The cells were then imaged on a Zeiss (Thornwood, N.Y.) LSM 510 confocal imaging system with an Axioplan 2 microscope (100× plan-apo objective, NA=1.4, oil) and a titanium sapphire multiphoton laser (mesochlorin, excitation 800 nm, emission 650 nm low pass filter; DAPI, excitation 800 nm, emission 461 nm band pass filter). The settings for all the confocal systems were adjusted so that control cells yielded dark images.

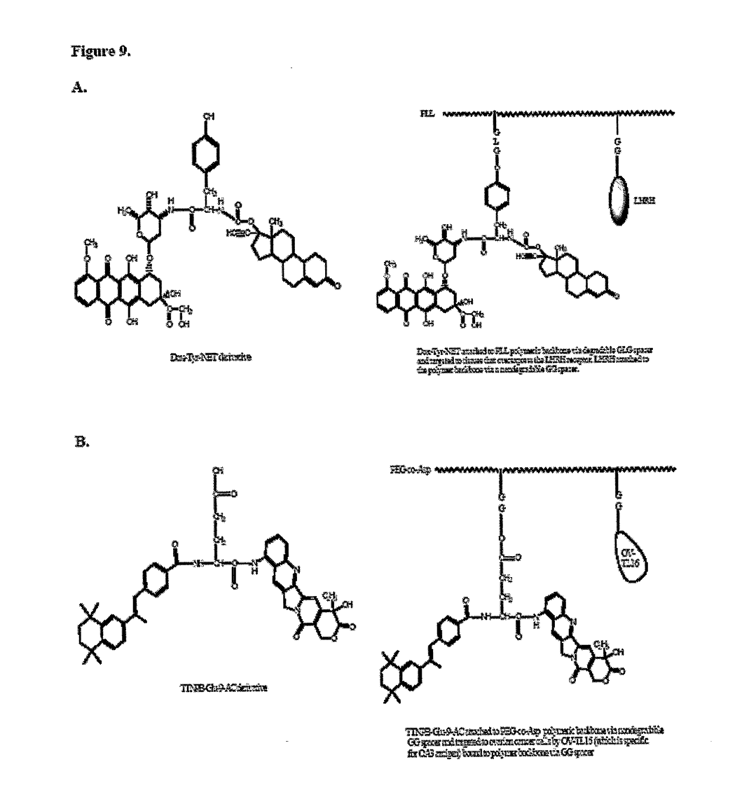

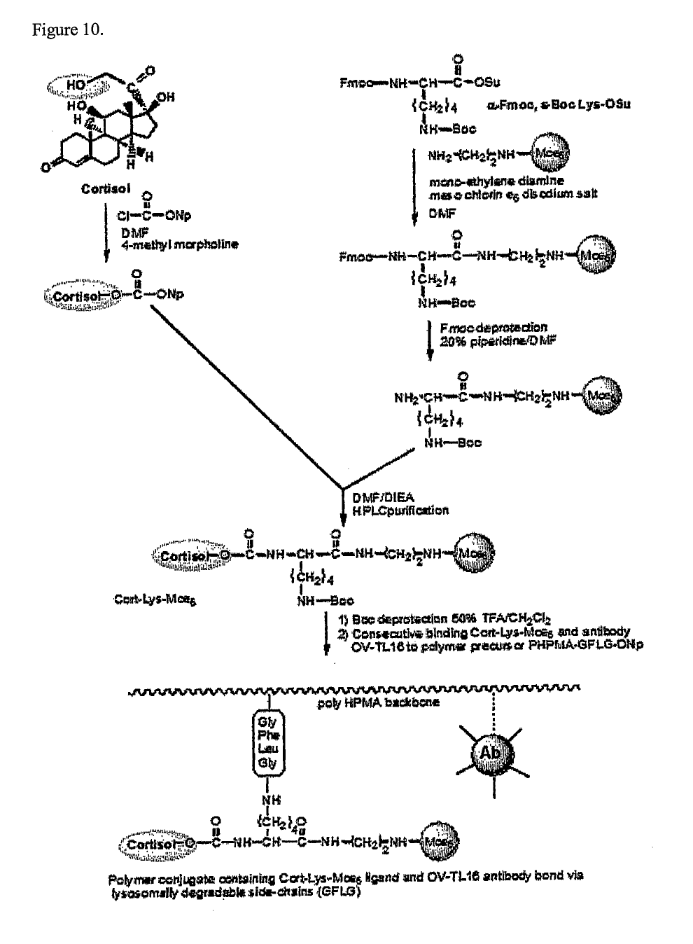

Results from the confocal microscopy experiments that were obtained are shown in FIGS. 9 and 10. In these images, DAPI is represented by green fluorescence. Hence, in all the images, the nucleus will be stained green in color. Though the staining is stippled, we can delineate the nuclear boundary and hence the nucleus from the stained areas. Mce₆ is stained red in color. Hence we can determine the subcellular distribution of Mce₆ in the cell by the red fluorescence. In both images (FIGS. 9 and 10), window A displays the fluorescence only due to Mce₆, while window B displays the fluorescence only due to DAPI. These two windows separately indicate the subcellular localization of Mce₆ and DAPI respectively. These 2 windows are superimposed in window D to display a composite image. In this composite image, we can demonstrate nuclear localization of Mce₆ if we can demonstrate the presence of red fluorescence (due to Mce₆) in the region of the nucleus (which will be delineated by the green fluorescence). Window C is a DIC image and is used to demonstrate the plane of visualization of the cells.

When the cells were treated with Mce₆, the red fluorescence was predominantly located in the cytoplasm. There was a faint red fluorescence due to Mce₆ in the nucleus (FIG. 9). In contrast, when the cells were incubated with Cort-Lys-Mce₆, Mce₆ was located in the cytoplasm and in the nucleus as demonstrated by the presence of the red fluorescence in both of these subcellular compartments. Colocalization of Mce₆ and DAPI demonstrated the nuclear localization of the Cort-Lys-Mce₆ (FIG. 10). We are thus able to demonstrate that the Cort-Lys-Mce₆ derivative localizes in the nucleus at much higher levels than free Mce₆ after incubation. This should translate into higher cytotoxicity values.

### Example VII

### Determination of Cytotoxicity

The IC₅₀ (concentration that inhibits growth by 50%) was determined utilizing a WST-8 assay. Four thousand cells were seeded into 96 well plates and incubated overnight in a humidified atmosphere containing 5% CO₂. Varying concentrations of free Mce₆ and Cort-Lys-Mce₆ or HPMA copolymer-bound Mce₆ conjugates (P-GFLG-Cort-Lys-Mce₆ and P-GFLG-Mce₆) were added to each well. Incubation was carried out for 4 hours in the case of free Mce₆ and Cort-Lys-Mce₆. The incubation period was 10 hours in the case of the HPMA copolymer-bound Mce₆ conjugates. After the period of incubation, the media containing the drug was removed and 100 μL of fresh media was added and the cells were promptly illuminated for 30 min. The light source was three ENH Tungsten halogen lamps (120 V/250 W) placed in parallel, attenuated by three band-pass interference filters (Melles Griot Co., Carlsbad, Calif.). The light energy was approximately 2 mW cm⁻² as determined using a radiometer.

After illumination, the cells were incubated in a humidified atmosphere containing 5% CO₂ for 24 hours. The media was replaced with 100 μL fresh media containing 10 μL of WST-8. After two hours incubation, the absorbance was read at 450 nm with background correction at 630 nm. Untreated cells served as 100% viable cells, whereas media served as background. Percentage of viable cells was calculated by dividing the mean absorbance of each well by the absorbance of the untreated cells. Linear regression was performed utilizing the linear portion of the growth inhibition curve to determine the IC₅₀ dose.

The values for the IC₅₀ dose after 4 hours of incubation with the drug that were calculated for Mce₆ and Cort-Lys-Mce₆ are shown in Table 1. The values in the table have been expressed in terms of Mce₆ equivalents. The data obtained reveals that Cort-Lys-Mce₆ is about 2.5 times more cytotoxic than free Mce₆.

IC₅₀ values calculated for P-GFLG-Mce₆ and P-GFLG-Cort-Lys-Mce₆ after 10 hours of incubation with the drug are shown in Table 2. IC₅₀ values have been expressed in terms of Mce₆ equivalents. Thus, P-GFLG-Cort-Lys-Mce₆ is about 2.5 times more cytotoxic than P-GFLG-Mce₆. These results demonstrate the increased efficacy obtained by targeting the nucleus with the drug using our system.

Thus, the invention provides nuclear targeting polymeric drug delivery systems based on HPMA copolymers wherein hormone analogs like cortisol are used as nuclear-targeting moieties. In vitro studies indicated that these systems are effective in achieving nuclear localization and increasing the efficacy of therapeutic agents.

### Example VIII

### Cytotoxicity Experiment

The details of the cytotoxicity experiment are as follows—SK-OV3 cells were plated at a density of 10,000 cells/well in 96 well plates. 24 hours after plating, the cells were incubated with varying concentrations of the drugs (both nuclear-targeted polymer-bound anticancer drug and non-nuclear targeted polymer-bound drug). Specifically, these drugs were cortisol-targeted HPMA-copolymer bound mesochlorin (the nuclear targeted construct) and HPMA-copolymer bound mesochlorin (the non-nuclear targeted construct). The drugs were incubated with the cells for varying durations. Following the period of incubation, the cells were washed, the medium was replaced and then irradiated for 30 minutes. 24 hours later, the cell viability was assessed using a modified MTT assay. In the best case example that was referred to in the previous email, the drugs were incubated with the cells for 10 hours. The cytotoxicity values that were obtained in this case were 110.00±7.07 micromolar for cortisol-targeted HPMA-copolymer bound mesochlorin (the nuclear targeted construct) and 3.38±1.33 micromolar for HPMA-copolymer bound mesochlorin (the non-nuclear targeted construct). This translates into approximately a 33-fold increase in cytotoxicity following nuclear targeting.

### Example IX

### Nuclear Localization

The details of the experiment to prove nuclear localization of the nuclear targeted construct are as follows—1471.1 cells were transfected with a plasmid (pCI-nGFP-C656G-Han) that causes the expression of Green Fluorescent Protein (GFP)-tagged Glucocorticoid Receptor (GR) using standard electroporation protocols. 24 hours after electroporation, the cells were plated in live cell chambers at a concentration of 200,000 cells/chamber. The cells were then treated with 100 micromolar concentration of cortisol-targeted HPMA-copolymer bound mesochlorin (the nuclear targeted construct) and HPMA-copolymer bound mesochlorin (the non-nuclear targeted construct). The fluorescence in each cell was followed using a fluorescence microscope. Cortisol-targeted HPMA-copolymer bound mesochlorin (the nuclear targeted construct) demonstrated a shift in fluorescence from a cytoplasmic distribution to a nuclear distribution within 1 hour of incubation. HPMA-copolymer bound mesochlorin (the non-nuclear targeted construct) did not show such a shift. This demonstrates that Cortisol-targeted HPMA-copolymer bound mesochlorin (the nuclear targeted construct) is taken up by the cells and results in nuclear targeting of mesochlorin due to interaction with the GR. Non-nuclear constructs do not show this property.

It is to be understood that the above-referenced embodiments are only illustrative of application of the principles of the present invention. Numerous modifications and alternative arrangements can be devised without departing from the spirit and scope of the present invention. While the present invention has been shown in the examples and is fully described above with particularity and detail in connection with what is presently deemed to be the most practical and preferred embodiment(s) of the invention, it will be apparent to those of ordinary skill in the art that numerous modifications can be made without departing from the principles and concepts of the invention as set forth herein.

Tables

TABLE 1
Cytotoxicity of Mce₆ and Cort-Lys-Mce₆ in murine
adenocarcinoma (1471.1) cells incubated for 4 hours
in DMEM with 10% charcoal stripped FBS

| Drug | IC₅₀ (μM) (Mean ± SD) |
|---|---|
| Mce₆ | 5.3 ± 0.93 |
| Cort-Lys-Mce₆ | 1.96 ± 0.17 |

TABLE 2
Cytotoxicity of P-GFLG-Mce₆ and P-GFLG-Cort-Lys-Mce₆
in murine adenocarcinoma (1471.1) cells incubated for
4 hours in DMEM with 10% charcoal-stripped FBS

| Drug | IC₅₀ (μM) (Mean ± SD) |
|---|---|
| P-GFLG-Mce₆ | 10.16 ± 0.19 |
| P-GFLG-Cort-Lys-Mce₆ | 4.17 ± 0.25 |

Appendix A. List of some receptors that can traffic to the nucleus and ligands that could be used to target them

| Receptor | Ligand |
|---|---|
| Androgen receptor (AR) | Testosterone |
| Progesterone receptor (PR) | Norethisterone |
| Estrogen receptor (ER) | Estrogen |
| Peroxisome Proliferation Activated | 15-deoxy-Δ 12,14-Prostaglandin J2 |
| Receptors (PPAR) | |
| Liver X receptors (LXR) | 24-OH cholesterol |
| Retinoid X receptors (RXR) | 9-cis retinoic acid |
| Benzoate X receptor (BXR) | 4-amino butyl benzoate |
| Constitutive Androstane Receptor | Androstanol |
| β (CARβ) | |
| Pregnane X receptor (PXR) | Pregnenolone 16-carbonitrile |
| Steroid and Xenobiotic receptor | Rifampicin |
| (SXR) | |
| Farnesoid X receptors (FXR) | 4-[(E)-2-(5,6,7,8-tetrahydro-5,5,8,8- |
| | tetramethyl-2-naphthalenyl)-1- |
| | propenyl]benzoic acid (TTNPB) |

All references, including publications, patents, and patent applications, cited herein are hereby incorporated by reference to the same extent as if each reference were individually and specifically indicated to be incorporated by reference and were set forth in its entirety herein.

1. A conjugate comprising a biologically active agent—subcellular targeting moiety complex.

2. The conjugate of claim 1, wherein the biologically active agent—subcellular targeting moiety complex is linked to a polymer by biodegradable or non-biodegradable spacers.

3. The conjugate of claim 2, wherein said polymer is further linked to a cellular targeting molecule biodegradable or non-biodegradable by spacers.

4. The conjugate of claim 1, wherein the subcellular targeting moiety is a steroid hormone or a steroid hormone analog.

5. The conjugate of claim 3 wherein the cellular targeting molecule is selected from the group consisting of ligand, polyclonal antibody, monoclonal antibody, phage display antibody, and ribosome display molecule.

6. The conjugate of claim 1, wherein the biologically active agent is selected from the group consisting of a drug, a prodrug, a gene, a nucleic acid sequence, a chemical compound, and mixtures thereof.

7. The conjugate of claim 2 wherein the polymer is a biodegradable spacer, and said biodegradable spacer is selected from the group consisting of an oligopeptide, spacers that undergo 1,6 elimination, pH sensitive bonds and disulfide bonds.

8. The conjugate of claim 7 wherein the biodegradable spacer is an oligopeptide selected from the group consisting of Gly-Phe-Leu-Gly (SEQ ID NO:1), Gly-Leu-Gly (SEQ ID NO:2), Gly-Val-Gly (SEQ ID NO:3), Gly-Phe-Ala (SEQ ID NO:4), Gly-Leu-Phe (SEQ ID NO:5), Gly-Leu-Ala (SEQ ID NO:6), Ala-Val-Ala (SEQ ID NO:7), Gly-Phe-Phe-Leu (SEQ ID NO:8), Gly-Leu-Leu-Gly (SEQ ID NO:9), Gly-Phe-Tyr-Ala (SEQ ID NO:10), Gly-Phe-Gly-Phe (SEQ ID NO:11), Ala-Gly-Val-Phe (SEQ ID NO:12), Gly-Phe-Phe-Gly (SEQ ID NO:13), Gly-Phe-Leu-Gly-Phe (SEQ ID NO:14), and Gly-Gly-Phe-Leu-Gly-Phe (SEQ ID NO:15).

9. The conjugate of claim 2, wherein the polymer is biodegradable in a lysosome.

10. A method of targeting a biologically active agent to the nucleus of a subject's cell, said method comprising:
administering the conjugate of claim 1 to the subject so as to target the biologically active agent first to the cell and then to the nucleus of the cell.

11. A method of concurrent nuclear and cellular targeting in a subject, the method comprising:
administering the conjugate of claim 1 to the subject for concurrent nuclear and cellular targeting.

12. A method of administering a biologically active agent to a cell comprising administering a steroid-targeted therapeutic to the cell.

13. The method according to claim 13 wherein the cell is cancerous.

14. An improvement in a conjugate comprising a drug linked to a polymer, the improvement comprising using a polymer biodegradable by an enzyme found in a cell lysosome.

15. The improvement of claim 14 wherein the polymer is an oligopeptide.

16. The improvement of claim 15 wherein the oligopeptide is selected from the group consisting of Gly-Phe-Leu-Gly (SEQ ID NO:1), Gly-Leu-Gly (SEQ ID NO:2), Gly-Val-Gly (SEQ ID NO:3), Gly-Phe-Ala (SEQ ID NO:4), Gly-Leu-Phe (SEQ ID NO:5), Gly-Leu-Ala (SEQ ID NO:6), Ala-Val-Ala (SEQ ID NO:7), Gly-Phe-Phe-Leu (SEQ ID NO:8), Gly-Leu-Leu-Gly (SEQ ID NO:9), Gly-Phe-Tyr-Ala (SEQ ID NO:10), Gly-Phe-Gly-Phe (SEQ ID NO:11), Ala-Gly-Val-Phe (SEQ ID NO:12), Gly-Phe-Phe-Gly (SEQ ID NO:13), Gly-Phe-Leu-Gly-Phe (SEQ ID NO:14), and Gly-Gly-Phe-Leu-Gly-Phe (SEQ ID NO:15).

17. The conjugate of claim 2, wherein the subcellular targeting moiety is a steroid hormone or a steroid hormone analog.

18. The conjugate of claim 3, wherein the subcellular targeting moiety is a steroid hormone or a steroid hormone analog.

19. The conjugate of claim 3, wherein the polymer is a biodegradable spacer, and said biodegradable spacer is selected from the group consisting of an oligopeptide, spacers that undergo 1,6 elimination, spacers having pH sensitive bonds, and spacers having disulfide bonds.

20. The conjugate of claim 3, wherein the polymer is biodegradable by a lysosomal enzyme.
